(19) United States
(12) Patent Application Publication
(10) Pub. No.: US 2020/0316129 A1
(43) Pub. Date: Oct. 8, 2020

(54) ENGINEERED ANUCLEATE CELLULAR AND EXTRACELLULAR VESICLES AS A NOVEL BIOLOGICS DELIVERY PLATFORM

(71) Applicant: THE JOHNS HOPKINS UNIVERSITY, Baltimore, MD (US)

(72) Inventors: Linzhao Cheng, Columbia, MD (US); Hao Bai, Lutherville-Timonium, MD (US); Vasiliki Machairaki, Baltimore, MD (US)

(21) Appl. No.: 16/303,829

(22) PCT Filed: May 25, 2017
(86) PCT No.: PCT/US2017/034360

§ 371 Date: Nov. 21, 2018

Related U.S. Application Data
(60) Provisional application No. 62/341,243, filed on May 25, 2016.

(51) Int. Cl. A61K 35/18; C12N 5/074; C07K 16/28; C12N 15/113; C12N 5/078

(52) U.S. Cl. CPC A61K 35/18; C12N 5/0696; C12N 2506/45; C12N 15/1136; C12N 5/0641; C07K 16/2863

(57) ABSTRACT

The present invention is based on the discovery of genetic engineered induced pluripotent stem cells (iPSCs) that produce one or more therapeutic entities such as antibodies and nucleic acids that when administered to a subject, will treat or prevent disease. The present invention also combines EMV technology with iPSC technology creating a powerful new drug delivery platform.

### REFERENCE TO RELATED APPLICATIONS

This application claims the benefit of U.S. Provisional Patent Application No. 62/341,243, filed on May 25, 2016, which is hereby incorporated by reference for all purposes as if fully set forth herein.

### GOVERNMENT INTEREST

This invention was made with government support under grant number HL 130676, awarded by the National Institutes of Health. The government has certain rights in the invention.

### BACKGROUND OF THE INVENTION

Human induced pluripotent stem cells (iPSCs), highly similar to embryonic stem cells (ESCs) that are able to expand in culture indefinitely while maintaining their full developmental potential, have been readily derived from somatic cells of patients and donors with unique genetic background. Recent development of reprogramming human blood mononuclear cells as well as fibroblasts to iPSCs, using improved methods such as by non-integrating plasmid vectors has been shown. The successfully reprogrammed iPSCs lack the epigenetic markers or signatures that are found in adult somatic cells and are associated with either cell specification/maturation or a disease state. For example, reprogrammed iPSCs from patient's fibroblasts of type I diabetes (mostly an epigenetic or non-mutated disease) behave similarly with iPSCs derived from normal controls, even in their mature progeny such as pancreatic and vascular cells after differentiation. Because of this and other unique properties, human iPSCs provide an unlimited and unprecedented source of autologous cell types for cell therapies of various diseases (with or without gene correction in iPSCs). In the past several years, scientists also developed a highly defined culture medium to expand human iPSCs on standard cell culture vessels coated with recombinant human vitronectin proteins (replacing feeder cells or matrigel). The defined cell culture medium, called E8, is based on DMEM and F12 basal media and free of any serum or animal proteins: with only 4 polypeptides (insulin, transferrin, bFGF and TGF31) of human or recombinant origin in the E8 medium. Evidence has been shown the E8 culture maintains pluripotency and genomic stability of human iPSCs after extensive culture. With this recent discovery, it is no longer a major hurdle to expand human iPSCs efficiently under a highly defined and clinically compliant culture condition. The highly defined and efficient iPSC culture system also makes it much easier to analyze unique sets of proteins, RNAs and metabolites that enable the unique and pluripotent capability of human iPSCs.

Unrelated to the field of iPSCs is a field of research on lipid bi-layered extracellular vesicles that are released by animal cells. These extracellular vesicles of either 50-200 nm in size (called exosomes) or 200 nm-1 μm in size (often called microvesicles) are membrane-bounded and anucleate. They may contain mRNAs, microRNAs and proteins. In this patent application, such exosomes and micro-vesicle collectively will be referred to as EMVs (others call them EVs or MVs).

EMVs have been found in biological fluids such as plasma and serum, and in conditioned medium of many animal cells in culture. For example, EMVs are found in commonly used fetal bovine serum (FBS) at a high level. EMVs found in human plasma and serum may have great impact on cell-cell communications. Therapeutic applications of novel IPSCs and EMV's must be identified to treat or prevent disease.

### SUMMARY OF THE INVENTION

The present invention is based on the discovery of genetic engineered iPSCs that produce one or more therapeutic entities such as antibodies, nucleic acids, fusion proteins, and peptides, that when administered to a subject, these therapeutic entities will treat or prevent disease. The present invention also combines EMV technology with iPSC technology creating a powerful new drug delivery platform. EMVs are created from genetically engineered iPSCs of the present invention that expression one or more therapeutic entities. EMVs of the present invention containing one or more therapeutic entity is then administered to a subject to treat or prevent disease.

One embodiment of the present invention is an induced pluripotent stem cell derived from a somatic cell, preferably an adult human somatic cell, which is preferably a blood mononuclear cell that expresses a therapeutic entity under the control of a HBB gene locus. The induced pluripotent stem cell is able to form exosomes including anucleated erythrocytes, platelets, and/or smaller microvesicles. An engineered induced pluripotent stem cell of present invention is able to express a therapeutic entity under the control of a HBB locus including a therapeutic protein or therapeutic microRNAs (miRs). A therapeutic protein or microRNA, when administered to a subject is able to treat or prevent a disease. Suitable therapeutic entities of the present invention include any gene expressing a therapeutic protein or a nucleic acid sequence that expresses a microRNA that may, by targeted insertion, be place under the control of the HBB gene locus in an induced pluripotent stem cell of the present invention. Examples include genes encoding proteins selected from the group consisting of sc-mAb targeting human IGFIR (insulin-like growth factor I receptor), sc-mAb targeting VEGFR2 (vascular-endothelial growth factor receptor 2), OCT4 (octamer-binding transcription factor 4), a single-chain monoclonal antibody blocking PCSK9 (Proprotein convertase subtilisin/kexin type 9), or a combination thereof. An example of a therapeutic entity that is a microRNA is Mir-302/367. It is preferred that the engineered induced pluripotent stem cells of the present invention produce anucleated erythrocytes comprising one or more therapeutic entity, specifically the protein or microRNA described above.

Another embodiment of the present invention is a method of generating anucleated erythrocytes comprising the steps of: culturing the induced pluripotent stem cells of the present invention; and generating anucleated erythrocytes from the induced pluripotent stem cells. This method preferably includes a step of collecting the anucleated erythrocytes comprising one or more therapeutic entity and placing them in a transfusion unit to enable the anucleated erythrocytes to be administered to a subject.

Another embodiment of the present invention is a pharmaceutical composition comprising an induced pluripotent stem cell of the present invention, an anucleated erythrocyte of the present invention, or both, and a pharmaceutically acceptable carrier.

Another embodiment of the present invention is a method of treating or preventing cancer in a subject comprising administering to the subject an effective amount of a pharmaceutical composition comprising induced pluripotent stem cells, anucleated erythrocytes, or a combination thereof that contain a therapeutic entity such as a sc-mAb targeting human IGF1R (insulin-like growth factor 1 receptor) that prevent or treat cancer, for example.

Another embodiment of the present invention is a method of treating or preventing ischemic retinopathy in a subject comprising administering to the subject an effective amount of a pharmaceutical composition comprising induced pluripotent stem cells, anucleated erythrocytes, or a combination thereof, that contain atherapeutic entity such as Mir-302/367, Myr-AKT, or a combination thereof, that can prevent or treat cancer, for example.

Another embodiment of the present invention is an induced pluripotent stem cell that expresses atherapeutic entity under the control of a HBB gene locus. It is preferable that the induced pluripotent stem cell forms an exosome, preferably in the size in the range of 1 μm or smaller though in some applications exosomes greater than 1 μm may be preferable. As describe above the induced pluripotent stem cells of the present invention are engineered to express one or more therapeutic entity such as a protein or nucleic acid sequence, such as a microRNA, that when administered to a subject is able to treat or prevent a disease. Suitable therapeutic entities are described above and within this specification. An engineered induced pluripotent stem cell of the present invention preferably produces anucleated exosomes comprising one or more therapeutic entity (ies).

Another embodiment of the present invention is a method of generating an exosome containing a therapeutic entity comprising the steps of culturing the induced pluripotent stem cells of the present invention; and generating exosomes.

Another embodiment of the present invention is a pharmaceutical composition comprising an induced pluripotent stem cell of the present invention, an exosome of the present invention, or a combination, and a pharmaceutically acceptable carrier. A pharmaceutical composition of the present invention may be used in a method of treating or preventing disease such as cancer or ischemic retinopathy, for example, in a subject, comprising administering to the subject an effective amount of a pharmaceutical composition of the present invention.

Another embodiment of the present invention is a method of preparing anucleated erythrocytes for drug deliver comprising the steps of providing an induced pluripotent stem cell derived from a blood mononuclear cell that expresses a therapeutic entity under the control of a HBB gene locus; generating an anucleated erythrocyte comprising the therapeutic entity; and collecting the anucleated erythrocytes and placing them in a transfusion unit.

Another embodiment of the present invention is a method of drug deliver comprising: providing an induced pluripotent stem cell derived from a blood mononuclear cell that expresses a therapeutic entity under the control of a HBB gene locus; generating an anucleated erythrocyte comprising the therapeutic entity from the induced pluripotent stem cell; and administering the anucleated erythrocyte to a subject.

Another embodiment of the present invention is a method of treating or preventing cardiovascular disease in a subject comprising administering to the subject an effective amount of the pharmaceutical composition of the present invention comprising anucleated erythrocytes comprising atherapeutic entity of a single-chain monoclonal antibody blocking PCSK9 (proprotein convertase subtilisin/kexin type 9.

The term “HBB” refers to Beta globin, also referred to as haemoglobin beta, hemoglobin bets, or preferably haemoglobin subunit beta, is a globin Protein. The HBB Protein is produced by the gene HBB. An example of a human HBB DNA sequence is NCBI database Gene ID: 3043. Assembly GRCh38.p7, and an example of a human HBB protein sequence is NCBI database accession number CAG46711.1.

The term “locus control region” or “LCR.” such as the locus control region of an HBB gene, refers to a long-range cis-regulatory element that enhances expression of linked genes at distal chromatin sites. It functions in a copy number-dependent manner and is tissue-specific, as seen in the selective expression of β-globin genes in erythroid cells.

The term “Sc-mAb” refers to a single-chain monoclonal antibody.

The term “subject” refers to any individual or patient to which the method described herein is performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

### BRIEF DESCRIPTION OF THE DRAWINGS

FIGS. 1A and 1B illustrates the expression of a heterologous protein (GFP) in iPSC-derived erythrocytes in coupling with HBB. (A) Targeted insertion of a marker gene (GFP) under the control of the HEB gene locus, without disrupting HB B protein production. See details of CRISPR-guide RNA (gRNA) mediated genome targeting at exon 1(1). Coding sequences (CDS) in three HBB exons are denoted in larger boxes, and un-translated 5′ and 3′ sequences in exon 1 and 3 are denoted in smaller boxes attached. A self-cleavable T2A peptide links the HBB, CDS, and the GFP reporter gene in frame. (B) Histogram of GFP expression in human erythrocytes differentiated from human iPSCs. The one from GFP-targeted iPSCs (shown in green curve) has a mean fluorescence intensity (MFI) of 60, and one from the unmodified iPSCs is shown in black, with MFI of 7.

FIG. 2 illustrates a diagram of a cell releasing microvesicles and exosomes, and a recipient cell up-taking these membrane-bound extracellular vesicles. MVE refers to multi-vesicular endosome.

FIG. 3 illustrates therapeutic applications of EMVs in regenerative medicine. Different EMV cargos, such as ATP and other small molecules, receptors such as VEGFR2, existing proteins and RNAs may be transferred into recipient cells and initiate a cascade of biological stimulation.

FIGS. 4A and 4 B illustrates characterization of EMVs from human iPSC line BC1, after purification and concentration (30 fold) by ultra-centrifugation. (A) Analysis of EMV size distribution and numbers by NanoSight, after 1:50 dilution. Red bars indicate one SD range. (B) A representative image of EMVs by TEM. Bilayer membrane-enclosed EMVs are evident.

FIG. 5A to SF illustrates FACS analysis of EMVs. (A) Human platelet-enriched plasma was used to set up a gating window. Red: platelets; Blue: residual cells (>3.2 μm). (B) EMVs from BC1 iPSCs. (C) Staining with Calcein AM (red) showing EMVs are metabolically active. (D). Surface staining with CD9 and CD63 are positive in the majority of EMVs. (E-F) Analysis of EMVs from the BC1-GFP iPSCs. The BC1-GFP EMVs (E) have a similar size profile as BC1 EMVs (B), but are positive for GFP signal in the majority of EMVs (green, in F).

FIG. 6A to 6C illustrates Luciferase activity detected. (A) The BC 1 iPSCs were stably transduced by the firefly luciferase (flue) gene. Various cell numbers (100-500) of harvested viable BCI (blue) and BCI-flue (red) were placed per well in 96-well plates (n=3). Then a D-luciferin containing solution was added to each well. Photon counts per second were measured within 15 min. (B) EMVs collected from BC1 or BC1-flue iPSCs and concentrated were measured similarly. (C) Live IVIS imaging by Xenogen after injection of EMVs into right eye of NSG mice (on the right). Mock-treated mouse was on the left. One day after, both animals were injected ip with D-luciferin and imaged.

FIG. 7A to 7B illustrates uptake and stimulated growth of ECs by EMVs from human iPSCs. (A) PKH26-labeled human iPSC-derived EMVs were incubated with ECs, and imaged after extensive wash. Image of PKH26 fluorescence (red) was over-laid with a phase-contrast image. (B) EMV stimulation of ECs. EMVs or the control E8 medium (ctrl) were added into a well containing 2000 ECs in 96-well plates in triplicates, EC growth was measured by WST-1 assay (OD450) at day 2 & 4.

### DETAILED DESCRIPTION OF THE INVENTION

Human erythrocytes (commonly called red blood cells or RBCs) are highly specialized small cells (5 μm) that lack nuclei, and able to circulate in the body for 120 days. They are widely used in transfusion medicine for treating chronic or acute anemia. It widely believed that young blood or plasma from young donors may have therapeutic effects for patients with age-related diseases; in fact, there are several ongoing clinical trials. However, it is challenging to obtain large amounts of blood or plasma for genetically matched donors without trigger immune rejection, especially after repeated or chronic transfusion. Recently, human erythrocytes were made in culture from adult human stem/progenitor cells (HSPCs), and successfully transfused back to a normal donor. However, this approach is limited by the current inability to expand human HSPCs, which is required to make industry-size erythrocytes. The present invention used human induced pluripotent stem cells (iPSCs) derived from adult somatic cells such as blood mononuclear cells to generate anucleate erythrocytes. The in vitro generated erythrocytes express high-level of hemoglobin, lack MHC membrane proteins as well as nuclei, and bind to oxygen efficiently. In addition, precise genetic modification aided by the CRIPSR-Cas9 system were made to correct a mutation in patient's iPSCs before differentiating them to functional erythrocytes.

The present invention is a novel drug delivery platform to deliver one or more therapeutic entities, such as polypeptides or nucleic acids, from the engineered iPSCs, and in vitro formed erythrocytes derived therefrom, upon being administered to a subject, and during their circulation in the body of the subject over their long-life span. Genetic engineering approaches (1) are used in the present invention to make better erythrocytes with gained biological functions, in addition to therapeutic benefits of erythrocytes carrying oxygen. For delivering a secreted protein or a nucleic acid, and to reach therapeutic effects, it is believed that only a fraction of engineered erythrocytes of the present invention need to be present in atypical RBC transfusion unit that would be used during a transfusion. In addition to erythrocytes, other anucleate membrane-enclosed vesicles could be made such as platelets (1 μm) or smaller micro-vesicles (<1 μm) or exosomes if they turn out to be a better choice for certain applications. The source of anucleate cellular vesicle could be autologous or from the IPSC lines with desirable genetic modifications.

The engineered anucleate cellular vesicles of the present invention, such as erythrocytes, platelets or smaller microvesicles is a novel platform for optimal and sustained delivery of various types of biologics as therapeutics for treating diseases such as cancers and chronic diseases. The therapeutic entity of the present invention maybe a therapeutic protein biologics including cytokines, enzymes, antibodies and soluble decoy receptors that are increasingly used in treating disease, which normally have higher specificity and reduced toxicity than chemical drugs. However, delivering these protein biologics to the right sites in patients to achieve an optimal and sustained dose is typically a challenge. Most polypeptidal biologics delivered by intravenous injection are often short lived in vivo, and repeated administration is often required. Based on our recent advances, a novel biologics delivery platform has been developed using therapeutic erythrocytes generated in vitro and in physiologic quantity from human iPSCs after genetic modifications.

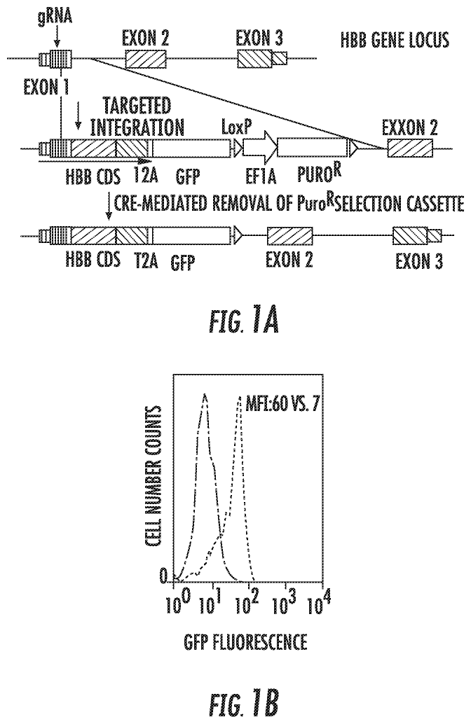

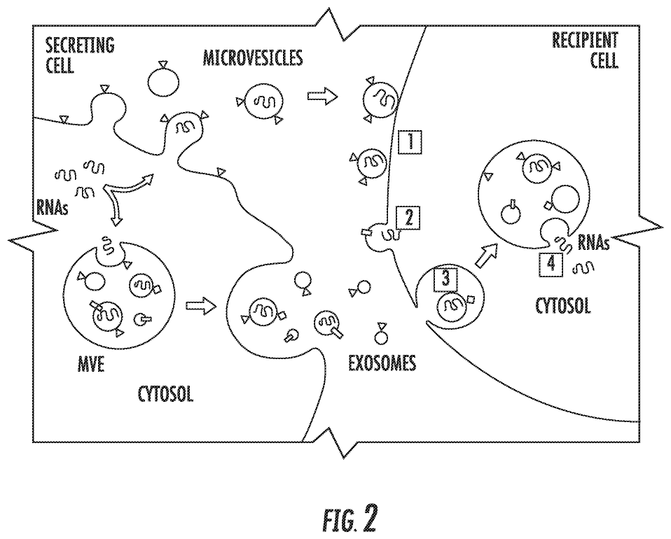

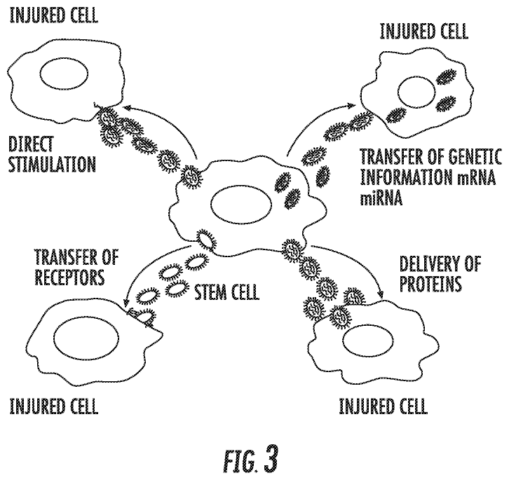

Human erythrocytes that express a soluble or membrane-bound protein targeting a human tumor xenograft in NSG immune-deficient mice will be developed. First erythrocytes will be generated in vitro from human iPSCs after CRISPR-Cas9 mediated correction of a mutation in the HBB gene, which encodes the adult form of hemoglobin beta subunit and mutated sickle cell disease and beta-thalassemia. iPSCs were produced that express a (marker) protein GFP under the locus control region of HBB gene (FIG. 1A), using the validated guide RNA targeting at exon 1. The HBB locus is chosen because HBB is one of the top 5 highly-expressed genes at the late stage of erythroid cells. The GFP marker gene is only expressed in late-stage erythrocytes (FIG. 1B). To demonstrate the feasibility, a tumor model was chosen that can be eliminated by a single-chain monoclonal antibody (sc-mAB) targeting IGF-1R (type 1 insulin-like growth factor receptor) that is highly expressed in a wide range of solid tumors and hematologic malignancies. More importantly, IGF-1R has been shown to be necessary for the transforming ability of several oncogenes. Therefore, it is not surprising that various reagents have been made including sc-mAb blocking this surface protein. For example, Amgen made Ganitumab (AMO 479) for various metastatic cancers. Although Amgen abandoned a Phase III trial in using Ganitumab for metastatic pancreatic cancers in August 2012, it still conducting a trial for treating newly diagnosed metastatic Ewing Sarcoma by combining chemotherapy with Ganitumab (NCT02306161). Investigators are still targeting this attractive surface protein using validated reagents in several studies. Such proteins can be engineered into iPSC cells, similar to GFP, as illustrated in FIG. 1, to create therapeutic iSPCs, platelets, micro-vesicles, and anucleated erythrocytes that comprise therapeutic entities. The therapeutic iSPCs, platelets, micro-vesicles, and anucleated erythrocytes may then be administered to a subject to deliver one or more therapeutic entity(ies) for purposes of treating or preventing disease.

The therapeutic iSPCs, platelets, micro-vesicles, and anucleated erythrocytes of the present invention may be used to treat or prevent cancer. It is well known that tumors and hematological cancers grow in a microenvironment enriched in blood vessels, even if cancer cells may tolerate a hypoxic condition much better than normal cells. The presence of tumor-associated neo-angiogenesis or neo-vasculogenesis is a hallmark of tumor growth. Although targeting tumor-associated neo-angiogenesis or neo-vasculogenesis using biologics has been attempted extensively in the past two decades, success is limited. One reason could be that biologics are often shortly-lived after intravenous injection. Using engineered erythrocytes of the present invention results in the ability to deliver these protein biologics more effectively and to achieve an optimal and sustained dose. One of the unique safety advantages of engineered erythrocytes of the present invention are that they lack nuclei (and the ability to replicate) and therefore are not tumorigenic. This is extremely important to novel cancer therapeutics.

However, this approach of using engineered erythrocytes or other anucleate biological vesicles of the present invention may be helpful in delivering therapeutic entities in subjects such as aflibercept or alemtuzumab (LEMTRADA), when necessary. This strategy of using engineered erythrocytes is especially attractive for treatment of cardiovascular diseases and malaria. When necessary, one can simply express the cDNA encoding alirocumab (Praluent®), a new single-chain monoclonal antibody blocking PCSK9 and reducing LDL-cholesterol in an induced pluripotent stem cell of the present invention.

Expressing a Secreted Protein from In Vitro Generated Erythrocytes

A strategy will be used to express a therapeutic entity in an erythrocyte as shown for the placement of a GFP gene into the HEB locus in human iPSCs and to express GFP in the in vitro generated erythrocytes from iPSCs (FIG. 1). The first choice of a secreted protein is the sc-mAb targeting human IGF1R that is highly expressed in a wide range of solid tumors and hematologic malignancies. An anti-IGF1R cDNA portion will be inserted into a targeting vector (to replace the GFP cDNA) and targeted into the HEB gene locus. This will be performed using the validated guide RNA targeting HBB and Cas9 expression vectors to target the established iPSC line that generate erythrocytes efficiently (1). We may add additional features to the targeted cDNA, such as making a fusion protein with GFP or luciferase as a tag to allow simple tracking of the production and circulation or with a functional protein to enhance anti-tumor effects. For the latter, our first choice is interleukin-12 (IL-12), which is a pleiotropic cytokine with potent immune stimulatory and anti-tumor activities but toxic to the host when being delivered systematically. A single cDNA encoding both IL-12 p35 and p40 subunits (available from InvivoGen) can be fused to the anti-IGFIR cDNA in frame. By measuring the tag (GFP, luciferase or IL-12), we will monitor the level of secreted proteins in the circulation or in the implanted tumor expressing high-level IGF1R.

The second (or an additional) choice of a therapeutic protein is the existing cDNA encoding a sc-mAb targeting VEGFR2 (vascular-endothelial growth factor receptor 2), able to block tumor angiogenesis or vasculogenesis. The overall approach would be similar to what we described above for targeting secreted anti-IGF1R sc-mAb in engineered human erythrocytes. The latest advance that disputing SH2B3 (LNK) gene may further enhance erythroid cell production may be utilized. The CRISPR-Cas9 system is amenable to target multiple genes (by simply adding additional guide RNAs to new targets), and much more efficient in disrupting a gene. We can deliver Cas9 to iPSCs as a riboprotein complex with guide RNA, instead of delivery a large plasmid DNA to express Cas9 protein. By this method, we are able to deliver multiple guide RNAs to target multiple sites, and also reduce Cas9-associated toxicity and off-targets. Note that the preferred final product of this approach is anucleate erythrocytes comprising one or more therapeutic entity (ies).

### Creation of Therapeutic EMVs

The present invention is focused on EMVs produced in large numbers from human iPSCs. We will use therapeutic EMVs of the present invention that are derived from iPSCs expressing one or more therapeutic entity (ies). The therapeutic or engineered EMVs of the present invention act as delivery vesicles for transferring native and heterologous proteins/RNAs, to subjects for therapeutic purposes (FIG. 3).

Human iPSCs Make a Great Number of EMVs Under a Highly Defined Culture Condition

Human iPSC were cultured and expanded using xeno-free, BSA-free E8 culture medium and use of recombinant human vitronectin substrate. Human iPSCs expand 2:10-12 fold in 3 days under either adherent or suspension culture conditions, but also derive new human iPSCs under completely defined culture condition with non-integrating plasmid vectors. We have examined EMV production from multiple human iPSCs including the BC1 line that was derived from a normal adult donor by non-integrating plasmid vectors, fully characterized for genomic integrity and used in differentiation for many cell types.

We collected daily the conditioned medium of iPSCs under standard culture condition. EMVs were purified by standard differential centrifugation to first remove cellular debris and aggregates, and later ultra-centrifugation at 100,000×g that also concentrates EMVs (9). After wash and re-spin at 100,000×g, the spun EMVs were resuspended in PBS (or other buffers or medium). We used standard methods to analyze these purified and concentrated EMVs.

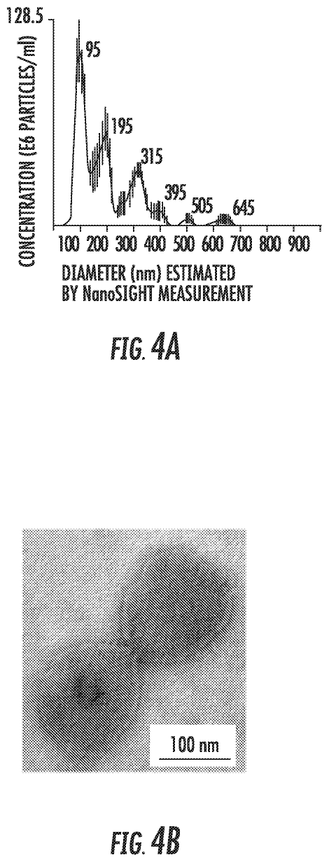

We used a NanoSight instrument (NS500 with a 405 nm laser) to conduct nanoparticle tracking analysis, estimating diameters of individual EMVs based on their Brownian motion, as shown in FIG. 4. It was found that human iPSCs produce a large number of EMVs containing multiple size groups including the major peak at 100 nm (FIG. 4a). The membrane-enclosed nature of EMVs was confirmed by visualization using transmission electron microscopy (TEM, FIG. 4b).

Using 30-fold concentrated EMVs (60.5×10⁹/ml), we estimate that the EMVs purified from 1 ml of human iPSC-conditioned medium contain about 7 ng and 4 ng total proteins and RNAs, respectively.

Similar data was also obtained with EMVs from multiple lines of human iPSCs by NanoSight: consistently we observed EMV densities at 2-4×10⁹/ml before any concentration steps. This was much higher than EMVs collected from other cell types such as human umbilical vein endothelial cells (HUVECs, 2.2×10⁷/ml). The total EMV particle concentration from human marrow-derived MSCs is similar to that from iPSCs. However, the standard human MSC culture medium contains 10% FBS as we and many others have been using in the past 15 years. Notably, EMVs from all sources are much more stable than their parental live cells in vitro: they can be stored short-term at 4° C. for weeks, or long-term at −20° C. or −80° C. without need for cryopreserving reagents such as DMSO. This unique feature (likely due to EMVs' small sizes) provides significant advantages over live cells in clinical applications.

Characterization of EMVs from Human iPSCs

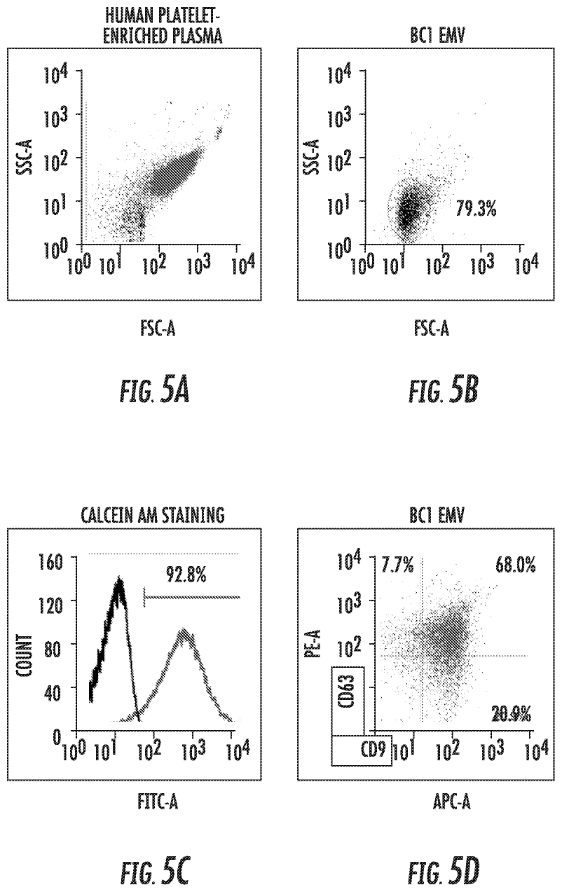

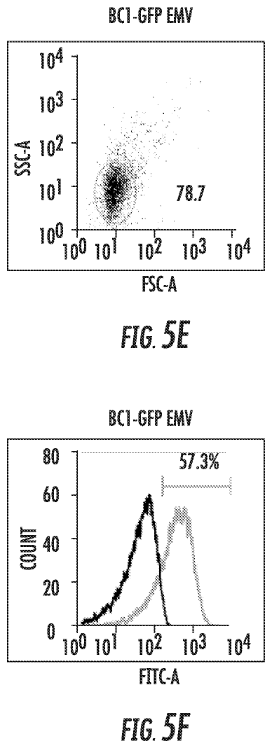

Using flow cytometric (FACS) analysis of small cellular vesicles such as erythrocytes and platelets, we used a FACS analyzer LSR II to analyze EMVs collected from human iPSCs (FIG. 5). Using an optimal setting of forward scatter (FSC-A) and side scatter (SSC-A), we can resolve EMVs from platelets (1 μm) and cells (>3.2-10 μm).

By FACS analysis, the size of most EMVs from human iPSCs have forward scatter (correlated to size) similar to that of recombinant lentiviruses (110 nm). They are smaller than platelets and residual cells (FIG. 5a) and 3.2 m calibrating beads (data not shown). The EMVs are metabolically positive and enclosed, because they are stained positive by a cytoplasmic esterase substrate, Calcein AM (FIG. 5c).

The 30× concentrated EMVs were stained by various specific monoclonal antibodies to detect surface expression of various protein antigens. We found that the majority of EMVs made by human iPSCs express CD9 and CD63 (FIG. 5d), known surface markers of EMVs and in iPSCs. The expression of CD81, MHC-class I (HLA-ABC) and E-Cadherin (data not shown), were detected as well as the other plasma membrane proteins known to be expressed in human iPSCs (and ESCs). The intensity of antigen levels in individual EMVs are lower than in individual cells, likely due to EMVs' smaller diameters and lower antigen numbers.

FACS analysis was used to determine if EMVs of BCI iPSCs that ectopically express GFP marker gene in cytoplasm also contain the GFP protein (FIG. 5e-f). In this BCI-GFP iPSC line we made, GFP transgene controlled by the CAG constitutive promoter is integrated in the genome and expressed in cytoplasm at a high level. The size profiles (and total particle numbers) of EMVs were very similar between those from BC1-GFP and parental BCI iPSCs (FIG. 5e). However, only the EMVs from BC1-GFP iPSCs displayed overt green fluorescence (FIG. 5f).

We have started systematically analyzing protein contents of human iPSC-derived EMVs using a recently improved proteomics based on mass-spectrometry. A preliminary analysis reveals the presence of membrane proteins such as CD81, CD90, and VEGFR2, known to be present in human ESCs and iPSCs. However, cytoplasmic proteins, and to a lesser extent nuclear proteins such as OCT4, were also detected. In addition to the PCR-based method that detected RNAs in iPSC-derived EMVs, we are also conducting systematic analysis of both mRNAs and small RNAs such as microRNAs (miRs) by RNA-Seq analysis. The preliminary analysis by RNA-seq of total RNAs confirmed that the OCT4, p-actin and the miR-302/367 cluster mRNAs were present in iPSC-derived EMVs.

EMVs from Human iPSCs Lack Genomic DNA but Contain RNAs

For this analysis, we used the EMVs collected from the BC1-GFP iPSCs after concentration of −100 fold. The EMV lysates were used either directly as a source of DNA template for PCR, or as a RNA source for reverse transcription (RT) followed by PCR. Three pairs of specific primers were used in quantitative PCR reactions, including the P-actin, GAPDH, and GFP genes (39). While transcripts were detected after RT-PCR, we did not detect DNA of the three genes under a sensitive detection condition (−1 copy of genomic DNA).

Additional Evidence that EMVs from Human iPSCs can Transfer a Heterologous Protein as Cargos.

To improve the sensitivity of cargo transfer, we use a BCI iPSC line that constitutively expresses firefly luciferase (fluc), after stable integration of a lentiviral vector. The enzyme fluc catalyzes a photon-generating reaction in the presence of its substrate D-luciferin, ATP and 0₂. D-luciferin is readily able to penetrate the membrane of animal cells both in vitro and in vivo.

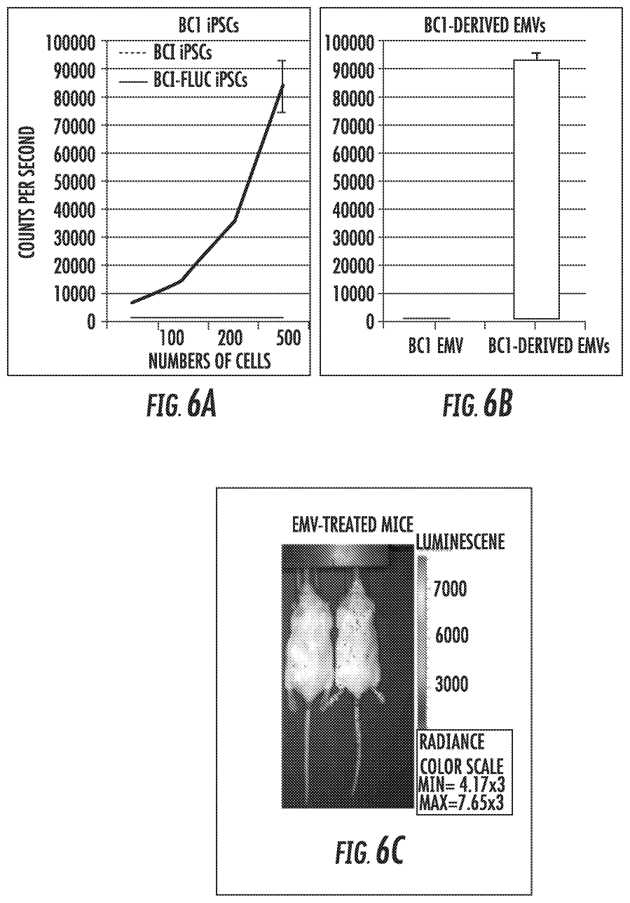

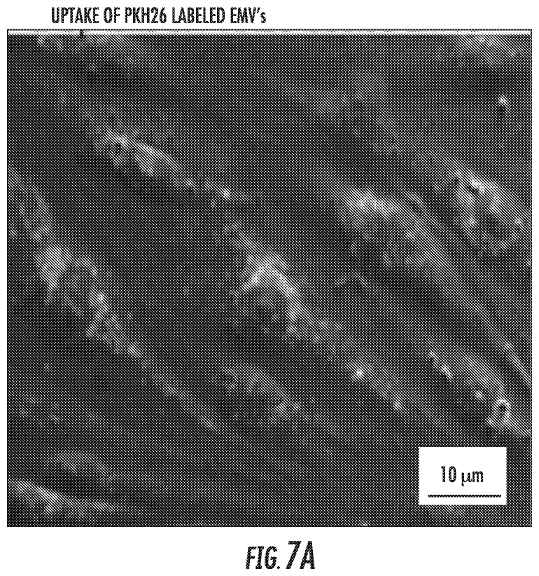

20 μl of 1OO× concentrated EMVs from BC 1-fluc iPSC cells was used and incubated with 100 μl solution containing D-luciferin (FIG. 6a, b). Flue luciferase activity equivalent to that within 500 viable cells was observed. Although it is difficult to determine the exact flue luciferase amount per EMV (because ATP and 02 level could be critically different in EMVs than in cells), it nonetheless indicates that EMVs are membrane-enclosed, and contain ATP (being metabolically active).

Moreover, up to 80 μl concentrated EMVs (˜100×) from BC1-fluc iPSC cells was injected into the retro-orbital vein of NSG mice, only in the right eye (with the mice face down). One day later, the presence of flue activity 15 min after intra-peritoneal D-luciferin administration was monitored (FIG. 6c). Clearly, flue activity only in the injected right eye of the treated animal (and maybe a low level in the circulation in the tail and legs) was observed.

The fates of flue-labeled EMVs was monitored as well as cells after intravenous or more localized injections, in live recipient animals non-invasively and serially. Together, the GFP+ and flue+ EMV data show that we can utilize EMVs to deliver a heterologous protein into target cells by producing it in cytoplasm of human iPSCs.

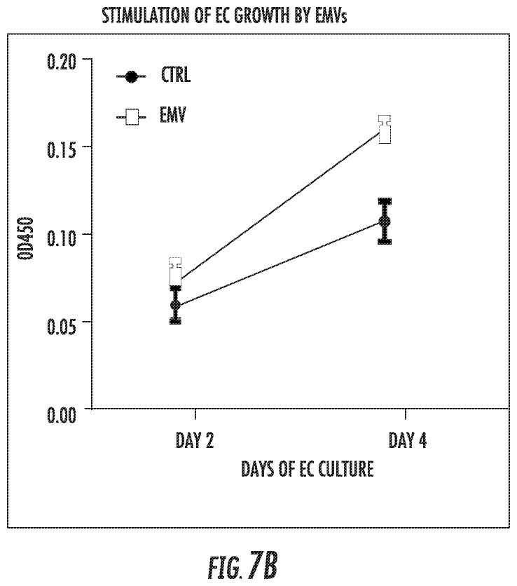

Uptake and Stimulation of Human Endothelial Cell Growth by EMVs of Human iPSCs (FIG. 7)

Could EMVs affect biological activities in vitro and in vivo? Endothelial cells (ECs) such as HUVECs and iPSC-derived ECs as target cells were used. To optimize the EMV uptake by ECs non-invasively and visually, purified EMVs were labeled with a lipophilic PKH26 dye that can integrate into the bi-layer lipid membrane of EMVs and emit red fluorescence.

After labeling of purified EMVs (also concentrated 100 fold by ultra-centrifugation) by PKH26 cell membrane labeling kit (Sigma), free forms of PKH26 dye were removed by a gel filtration column to exclude small molecules of: 3,000 Da. The PKH26-labeled EMVs from human iPSCs were added into the medium of EC culture. Twenty hours after incubation, the ECs were washed extensively and visualized by fluorescence microscopy (FIG. 7a). It is evident that PKH26-labeled EMVs were internalized by ECs, and found especially in peri-nuclear membrane areas and other membrane-enriched regions (where membrane-bound PHK26 displayed the strongest signal).

Encouraged by the uptake result, the growth of ECs treated with EMVs from iPSCs or concentrated medium control were monitored (FIG. 7b). The growth of ECs was monitored by the standard WST-1 cell growth assay, 2 or 4 days after adding the EMVs. Significant enhancement was first observed at day 2, and was more evident at day 4. Together with other preliminary data, the detected biological activities of EMVs from human iPSCs encourage us to conduct a systematic characterization of EMVs, and investigate their in-vivo potential.

While many investigators in the EMV field are working on whether EMV-containing proteins and RNAs could be better biomarkers to monitor pathologic processes, the present invention is focused using human EMVs produced from stem cells such as iPSCs as delivery vesicles for transferring native and heterologous proteins/RNAs and for therapeutic purposes. Anucleate EMVs released from cells provide a different cell-free approach complementary to ongoing research using virus-like particles or other membrane-enclosed nano-particles for delivering biologics in vivo.

So far, the publications on therapeutic applications of human EMVs are mostly using human MSCs derived from marrow, adipose, umbilical cord, or differentiated iPSCs. The latter two types of MSCs are increasingly used because they have greater and longer cell proliferation and/or can generate more EMVs. Until recently, human MSCs are the easiest non-transformed human cell types to culture and expand (at least in the presence of 10% FBS that provides nutrients and extracellular matrix proteins). Additional features, such as being a rich source of trophic factors, unique immune-suppressive activities and extensively attempted clinical trials for various cell therapeutics, may also drive MSC's popularity as a source of EMVs. While it is important for us to follow ongoing progress using human MSC-derived EMVs and use them in our research as important controls for comparison, the present invention is focus on the relatively unexplored use of human iPSC-derived EMVs.

Human iPSCs, and EMVs generated from human iPSCs in cultures, have several unique features. In addition to the key technological advantage that iPSCs can be easily expanded in the defined E8 medium (serum-free and xeno-free) and that they produce large numbers of EMVs, iPSCs also have additional unique biological properties. They have extremely great self-renewing capability in culture, while maintaining their pluripotency. They express unique sets of mRNAs and proteins such as OCT4, that are also present in the derived EMVs. Similarly, human iPSCs and derived EMVs also contain unique miRs such as the miR-302/367 cluster, with unique biological functions. We will investigate the ability of EMV-mediated transfer of self-renewing and reprogramming regulators such as OCT4 and miR-302/367, either as the endogenous forms or over-expressed forms derived from human iPSCs, to “rejuvenate” somatic cells such as ECs after injury or under a disease state.

Human iPSCs (as well as ESCs) also have unique metabolic activities. Reprogramming human somatic cells to iPSCs results in a contraction of the mitochondrial DNA content, and a switch from oxidative phosphorylation to glycolysis . . . . As a result, endogenous levels of reactive oxygen species (ROS) in human iPSCs are much lower than those in other cell types such as MSCs in culture.

Human EMVs, especially those from human iPSCs with genetic modification, are a new way for delivery of biologics via anucleate extracellular vesicles. With our expertise in precise genome editing and genetic modification of human iPSCs, we are able to make genetically-enhanced EMVs that carry heterologous proteins and RNAs resulting from over-expression in parental human iPSCs. As compared to other anucleate (and extracellular) vesicles that we have worked with (such as platelets that are 1 μm in size and made only from megakaryocytes in mammals), we have determined that EMVs can be formed by almost any cell types including human iPSCs. EMVs from human iPSCs can be generated without a lengthy differentiation process (21 days) to generate megakaryocytes and then pro-platelets as we reported. We envision that human iPSC-derived EMVs as anucleate biologics provides a novel modality of “cell” therapies without the need to transfuse or transplant nucleated cells that have risk of over-proliferation and cancer formation.

Properties & Biological Activities of EMVs from Human iPSCs and MSCs

EMV densities of −2-4×10⁹ per ml of daily collected supernatant (conditioned medium) from sub-confluent human iPSCs and MSCs, before any concentration steps were consistently observed. However, the exact number of MSC-derived EMVs is difficult to determine, because we used the standard MSC medium containing 10% FBS that contributes a larger number of bovine EMVs and varies from batch to batch. In contrast, we culture human iPSCs with a highly defined E8 medium without any animal proteins or serum/plasma. For human MSCs, we will first collect EMVs after overnight culture in E8 medium (after wash to reduce residual FBS components). If the total MSC-EMVs collected in E8 medium are too low (5-10 fold) in number or there are still too many bovine EMVs (210% of total), the inventors will use Life Technologies' StemPro MSC/SF medium (not using FBS or human plasma platelet lysates but containing BSA fraction V) or a xeno-free version

The inventors analyze EMVs from both human iPSCs and MSCs (collected in defined media) before and after purification/concentration, using the standard methodology and described in the preliminary data (FIG. 4-7). Additional assays include using flow cytometry to measure levels of surface phosphatidylserine (PS, an inflammatory signal), and ROS in EMVs from human iPSCs and MSCs. For the latter, we will incubate EMVs as well as their parental cells with di-chloro-fluorescein-diacetate (DCFDA) or MitoSox. Alternatively, we will use di-hydro-rhodamine (DHR) as we used before in a neutrophil study, especially if we need to analyze GFP-labeled EMVs. We will determine if the endogenous ROS level in iPSC-derived EMVs is indeed much lower than that in MSC-derived EMVs, likely due to the fact that iPSCs primarily use glycolysis.

If numbers of EMVs are too low for this project even after concentration, we will explore simple ways to enhance EMV production from human MSCs and iPSCs. We will start with simple means such as changing oxygen content (from 20% to 5%), adding ATP into culture, etc. If necessary, we will conduct a small-scale screening of small molecules (3600 in the Hopkins Clinical Compound Library) or Sigma's LOPAC (1280 compounds) that are both available at Hopkins' ChemCore facility. A flue-based assay by a 96-well plate-reader (FIG. 6) will allow us to have a quick screening of EMVs collected from treated cells (in triplicates) cultured in 96-well plates. We will select the treatment that significantly increases EMV production (2:5×), without increasing ROS levels. Once basic culture conditions for large-scale EMV production by human MSCs as well as iPSCs are established, we will also conduct RNA-seq and proteomics of EMVs and their parental cells. Together with MSCs and iPSCs from other donors, the direct comparison of RNA expression in EMVs derived from iPSCs (BC1 and E2) with that of MSCs of the same donor will reduce Variations resulting from genetic polymorphisms among different human donors.

Human iPSC-derived EMVs enhanced the growth of human endothelial cells (ECs) such as HUVECs and iPSC-derived ECs was observed (FIG. 7). It is well established that VEGF present in the EC culture medium is critical for survival and proliferation of ECs, which express VEGF receptor 2 (VEGFR2, also known as KDR and FLK1). In preliminary studies, human iPSC-derived EMVs also expressed VEGFR2, which are also expressed in 20 human iPSCs and ESCs. To test the hypothesis that EMVs mediate a transfer of receptors such as VEGFR2 and thus stimulate EC growth, we will conduct the following experiments and use the same HUVECs and iPSC-derived ECs as we did before, as well as human retinal endothelial cells (HRECs, relevant to the eye disease model we will describe later). We will conduct both loss-of-function and gain-of-function experiments. For loss-of-function, the inventors will knock out or knock down VEGFR2 expression in human iPSCs. VEGFR2 appears to be dispensable for the growth of human iPSCs and ESCs (our unpublished data). We will also find out if the absence of VEGFR2 receptors on human iPSCs is essential to EMV formation.

If not essential, the inventors will use VEGFR2-negative EMVs for incubation with ECs, to examine their stimulatory activities on EC growth. As an additional negative control, we will use EMVs from MSCs that are supposed to be negative for VEGFR2 expression.

For gain-of-function experiments, we will over-express the human VEGFR2 gene in human iPSCs (based on a VEGFR2-mEmerald RFP fusion protein encoded by the Addgene plasmid #54298), as we did previously with GFP and flue reporter genes in human iPSCs. The level of VEGFR2 expression in EMVs will be determined by FACS with a specific antibody that we used before. We may be able to monitor the transfer of VEGFR2 to target ECs by monitoring mEmerald RFP.

MicroRNAs (miRs) are 18-24 nucleotide (nt)-long noncoding RNAs that are synthesized as a longer RNA precursor (pri- and pre-miR) from a RNA polymerase II or III promoter. They bind to partially complementary sequences (18-nt or short) present in mRNAs of multiple genes, cleaving mRNAs or inhibiting their translation. Recent studies demonstrate that miRs play important roles in human ESC and iPSC self-renewal and somatic cell reprogramming. Among them, the miR-302/367 cluster is highly expressed in human ESCs, but is much lower in adult somatic cells such as HUEVCs and fibroblasts. Overexpression of this miR cluster can promote the growth of human ESCs and iPSCs, and somatic cell reprogramming to iPSCs. In human ESCs and iPSCs, the miR-302/367 cluster blocks cell cycle inhibitors and conquers apoptosis by down-regulating BNIP3L/Nix. In somatic cells that lack miR-302/367, expressing the 4 reprogramming factors (OCT4, SOX2, KLF4 and Myc) and other reprogramming treatment (such as adding butyrate) significantly activates the transcription of pri-miR-302/367 RNA from the endogenous Pol II promoter containing the OCT4 and SOX2 binding sites. This is consistent with the observation by many that over-expression of miR-302/367 is able to enhance the reprogramming of somatic cell types even in the absence of butyrate.

Our preliminary RNA-seq analysis and a PCR-based assay indicate that miR-302/367 cluster and its 5 mature family members miR-302a, 302b, 302c, 302d and miR-367 are abundantly expressed in iPSC-derived EMVs as in the parental iPSCs. Because the expression level of miR-302/367 members are very low in adult (non-transformed) somatic cells such as ECs and fibroblasts (44-45; 49-50), we believe that iPSC-derived EMVs stimulate EC growth by: 1)transferring mature miR-302/367 members and exerting biological functions by targeting multiple mRNAs; and/or 2) activating endogenous miR-302/367 gene expression via iPSC-specific factors such as OCT4 (both RNA and protein) that were also detected in the iPSC-derived EMVs. We will be able to distinguish between the two possibilities and estimate the importance of each route (exogenous vs. endogenous MiR-302/367 effects), although it could be the combination of both.

To determine the expression level of (endogenous) miR-302/367 cluster members in ECs, we will measure the precursor (pri- and pre-miR) RNA level by PCR using primers specific to the sequence portion not present in the mature miR members that are delivered by EMVs. RNAs from human iPSCs will be used as a positive control and untreated ECs will be used as a negative control. The stimulation of the expression of the endogenous miR-302/367 cluster can be further confirmed by using a flue reporter under the control of the miR-302/367 promoter. If activation of the endogenous miR-302/367 is negligible, then we will focus on the exogenous miR-302/367 (mature) members for the biological effects, especially immediately after the EMV delivery to ECs. Moreover, biological functions of EMV-delivered exogenous miR-302/367 (mature) members could be further assessed by using human ESCs and iPSCs in which the miR-302/367 cluster is inducibly suppressed. Necessary reagents include human iPSC lines with inducible knock-out of the miR-302/367 cluster, a flue-based promoter reporter, and expression vectors to over-express miR-302 and miR-367 mature forms.

The knowledge obtained above will be important to design experiments and time-courses to narrow down the major target mRNAs of miR-302/367-mediated suppression of translation. Previously reported target mRNAs include those encoding cell cycle progression inhibitors, and the TGFβ/activin/nodal and the PI3K/AKT pathway components and regulators. When we analyze the effect of miR-302/367 on the protein levels of multiple cell cycle inhibitors, we will also analyze cell cycle status by the standard methods after EMV treatment. In addition to cell cycle regulation, we will focus on the impact of miR-302/367 on these two key signaling pathways. We observed an additional stimulation of EMVs on EC growth in the presence of an inhibitor of TGFβ/activin/nodal signaling pathway, SB431542, which itself prevents EC senescence and stimulates their growth. Therefore, the inhibition of TGFβ/activin/nodal signaling pathway is unlikely to be a key target of the miR-302/367 stimulation on ECs. The direct effects of miR-302/367 on the PBK/AKT signaling pathway may be cell-context dependent. There is a canonical, perfect-seed binding site for the miR-302 family in the 3′ UTR of the PTEN mRNA, a negative inhibitor of the PBK/AKT signaling pathway that is known to universally enhance cell survival and proliferation (including ECs) after receptor activation. We will also be able to modulate the axis of miR-302/367 and PBK/AKT signaling by transgenic approaches below.

We have demonstrated that we can overexpress mRNAs encoding cytoplasmic proteins such as GFP and flue in human iPSCs, which can be packaged into EMVs as cargos and delivered passively into cytoplasm of target cells. We will be able to over-express membrane proteins such as VEGFR2, which will be packaged in EMVs and horizontally transferred to the membrane of the target cells. We will be able to over-express other heterologous proteins in EMVs, especially by tethering them to the cell membrane in order to accumulate to a high concentration in EMV s and target cells. To establish such a robust platform for various proteins, we will take the approach of tethering a heterologous protein, through genetic engineering, to a myristoylation (myr) signal peptide that enables the protein to be anchored on the inner plasma membrane. Upon receptor activation, cytoplasmic AKT proteins are myristoylated, move close to inner membrane and be activated by lipid-dependent PB kinase. To express a myr-AKT protein in human iPSCs, we will first use an existing vector expressing a myr-HA-AKT1 fusion protein (also HA-tagged) that is available from Addgene (plasmid #48969 for a lentiviral vector pCDH-puro-myr-HA-Aktl). The transgene only contains AKTJ coding sequence and is resistant to possible suppression by miR-302/367 that targets the 3′ UTR in AKT1 mRNA. The expression of myr-AKT1 protein may enhance the EC survival and growth by EMVs. If necessary, we may use the coding sequence of the AKT2 gene, which has similar biochemical functions.

We will also use over-expression vectors that express a miR-302 mature member and miR-367. Based on the available expression vector, we will co-express miR-302b (that share the identical target sequence as other miR-302 members) and miR-367 mature form in human iPSCs. When necessary, we will also conduct gene knock-out using the CRISPR-Cas9 technology in human iPSCs, used in our previous studies. The capability of generating genetic modified EMVs will further enhance not only our mechanistic studies, but also desirable biological functions of EMVs. The established principle and methodology should also be applicable to other miRs such as a proangiogenic miR-132 that is expressed at low level in both ECs and iPSCs, and various mRNAs that encode antigenic proteins. We have developed a reliable and efficient technology for transfecting RNAs (even proteins) into colloidal EMVs, and we are also able to deliver exogenous biologics (proteins and RNAs) directly into EMVs.

We will inject concentrated and purified EMVs into one eye intravenously (via retro-orbital vein) as we did in FIG. 6c or intravitreously. The live imaging of whole mice will be conducted daily in the next 3 days, and then day 6-7 and day 14. We can also change the setting of Xenogen IVIS imaging system to focus on the head of animals and distinguish signals in two different eyes. We will establish a baseline of EMV bio-distribution in NSG 15 mice, before we will use genetically enhanced EMVs such as those over-expressing miR-302/367 and/or myr-AKT that are able to enhance EC growth, and stimulate angiogenesis. It is likely that EMVs injected intravitreously will stay longer and yield stronger signals locally in the injected eye than intravenous injection. However, imaging of EMV distributions after intravenous injection is also important, which provide a control to assess EMV's presence (PK/PD) in the circulation system and their preference to migrate and lodge in different organs. The flue-imaging results will be backed by the effort to measure human EMVs in mouse blood after intravenous injection, using a FACS-based assay we developed (FIG. 5) and human-specific antibodies such as those recognizing human CD9 and CD63.

Even if the inventors no longer detect flue signals after 7-14 days, we will monitor closely the injected animals by naked eyes for up to 4 months. The main purpose is to examine the safety of EMV injection. If an adverse effect occurs, a mouse pathologist in the animal core will examine pathology.

If we detect flue signal in the injected eye after EMV delivery such as one by intravitreal injection, we will conduct more vigorous biological studies in vivo. An emphasis will first placed on ECs and vasculature. Similar experiments will also be conducted later using EMVs expressing miR-302/367 and/or myr-AKT.

We will first use a validated mouse model that is commonly used by many eye researchers. We chose this model of eye disease, based on experimental and future clinical trial considerations. Ocular treatment in one eye at a time offers several advantages in assessing safety and efficacy including the fact that the untreated eye provides an ideal control. Smaller numbers of EMVs are required for ocular injection and treatment. Eyes are also considered as an immune privileged site that has reduced immune responses to neo-antigens associated with culture-produced cells or EMVs.

Ischemic retinopathies including diabetic retinopathy and retinopathy of prematurity (ROP) are major causes of blindness in the U.S. and globally. The mouse model of oxygen-induced retinopathy (OIR) is a well-studied model of ischemic retinal diseases. This because the OIR model shares the features of ischemia and pathologic pre-retinal NV in human patients. The pathologic pre-retinal NV can cause a maladaptive response, and in humans ultimately can lead to vitreous hemorrhage and tractional retinal detachment that can result in blindness.

In this mouse model, newborn mice are treated with 75% oxygen at postnatal day 7 (P7) for 5 days. Avascular retina is evident at day 12, when mice are returned to normal oxygen condition (normoxia). At this stage, vessel loss triggers atypical hypoxic response and production of vascular and pro-angiogenic factors such as VEGF, which in turn result in aberrant endothelial cell proliferation seen at P12 and pathologic pre-retinal NV seen at P17. Notably, between P12 and P17, certain degrees of reparative angiogenesis of the retina does occur, although the amount is only modest, so that there is still significant retinal ischemia that drives pathologic pre-retinal NV. It would be highly desirable in this OIR model (and in corresponding human ischemic retinal diseases including diabetic retinopathy and ROP), if a greater degree of reparative angiogenesis of the ischemic retina could occur, which would improve recovery of the normal retinal tissue, reduce retinal ischemia and consequent production of pro-angiogenic factors, and reduce pathologic pre-retinal NV. We will inject EMVs intravitreously into one eye of the treated animal starting at PIO. The effect will be monitored at P12, P17 and P25 in the treated and untreated eye of the same animal and compared with the control animal group (no hyperoxic treatment). If necessary, we will inject EMVs again at P12. We will also test genetically enhanced EMVs (such those over-expressing Mir-302/367 or Myr-AKT), in comparison with unmodified EMVs from human iPSCs and MSCs. The vasculature of the eyes treated by various EMVs will be compared with, following the established protocol.

Embodiments of the disclosure concern methods and/or compositions for treating and/or preventing disease such as cancer, ischemic retinopathy, or cardiovascular disease, as examples, using induced pluripotent stem cells, anucleated erythrocytes, or anucleated exosomes comprising a therapeutic entity of the present invention, collectively called therapeutic agents of the present invention. In certain embodiments, individuals with one or more of these disease are treated with one or more therapeutic agents of the present invention. In a specific embodiment, an individual with ischemic retinopathy is provided an induced pluripotent stem cells, anucleated erythrocytes, or anucleated exosomes comprising a microRNA Mir-302/367, Myr-AKT (myristolated active form of AKT) or a combination thereof such as to treat or prevent ischemic retinopathy.

In particular embodiments of the disclosure, an individual is given an agent for treating or preventing a disease in addition to the one or more therapeutic agents of the present invention. When combination therapy is employed with one or more therapeutic agents of the present invention, the additional therapy may be given prior to, at the same time as, and/or subsequent to the one or more therapeutic agents of the present invention.

### Pharmaceutical Preparations

Pharmaceutical compositions of the present invention comprise an effective amount of one or more therapeutic agents of the present invention, dissolved or dispersed in a pharmaceutically acceptable earner. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one or more therapeutic agents of the present invention or additional active ingredient will be known to those of skill in the art in light of the present disclosure, as exemplified by Remington: The Science and Practice of Pharmacy, 2 nd Ed LipP.incott Williams and Wilkins, 2005, incorporated herein by reference. Moreover, for animal (e.g. human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g. antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference) Except insofar as any conventional carrier is incompatible with the active ingredient, its use in pharmaceutical compositions is contemplated.

The one or more therapeutic agents of the present invention may comprise different types of carriers depending on whether it is to be administered in solid, liquid or aerosol form, and whether it need to be sterile for such routes of administration as injection. The present compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The one or more therapeutic agents of the present invention may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid Salts formed with the free carboxyl groups can also be derived from inorganic bases such as for example, sodium, potassium, ammonium, calcium or ferric hydroxides, or such organic bases as isopropylamine, trimethylamine, histidine or Procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present invention suitable for administration is provided in a pharmaceutically acceptable carrier with or without an inert diluent. The carrier should be assimilable and includes liquid, semi-solid, i.e., pastes, or solid carriers Except insofar as any conventional media, agent, diluent or carrier is detrimental to the recipient or to the therapeutic effectiveness of a composition contained therein, its use in administrable composition or use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens, chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art. In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include one or more therapeutic agents of the present invention, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the one or more therapeutic agents of the present invention may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram kg/body weight, about 100 milligram/kg/body weight, about 200 milligram/kg/body weight, about 350 milligram/kg/body weight, about 500 milligram/kg/body weight, to about 100 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Alimentary Compositions and Formulations

In one embodiment of the present disclosure, the one or more therapeutic agents of die present invention are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following, a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See. e.g., U.S. Pat. No. 5,629,001. Upon reaching the small intestines, the basic pH therein dissolves the coating and permits the composition to be released and absorbed by specialized cells. e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparations and formulations.

Oral administration of the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed n a dentifrice, or added in a therapeutically-effective amount to a composition that may include water, binders abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### Parenteral Compositions and Formulations

In further embodiments, one or more therapeutic agents of the present invention may be adminstered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmaceutically acceptable salts may be prepared in water suitably mixed with a surfactant, such as hydroxypropylcellulose Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propyleneglycol, and liquid polyethylene glycol, and the like), suitable mixtures thereof and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, ages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by IFDA Office of Biologics standards.

Sterile injectable solutions are prepared by incorporating the active compounds in the required amount in the appropriate solvent with several of the other ingredients enumerated above, as required, followed by filtered sterilization Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### Miscellaneous Pharmaceutical Compositions and Formulations

In other preferred embodiments of the invention, the active compound or one or more therapeutic agents of the resent invention may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and laurocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of compositions of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements m all possible variations thereof is encompassed by the invention unless otherwise indicated herein or otherwise clearly contradicted by context.

1. An induced pluripotent stem cell derived from a blood mononuclear cell that expresses a heterologous therapeutic entity protein or nucleic acid sequence under the control of an endogenous gene locus.

2. The induced pluripotent stem cell of claim 1 that forms an anucleated erythrocyte.

3. The induced pluripotent stem cell of claim 1 wherein the therapeutic entity nucleic acid is a miR.

4. The induced pluripotent stem cell of claim 1 wherein the therapeutic entity is a protein selected from the group consisting of sc-rnAb targeting human IGF IR (insulin-like growth factor 1 receptor), sc-mAb targeting VEGFR2 (vascular-endothelial growth factor receptor 2), OCT4 (octamer-binding transcription factor 4), a single-chain monoclonal antibody blocking PCSK9 (Proprotein convertase subtilisin/kexin type 9), Myr-AKT (myristoylated, active form of AKT), and a combination thereof.

5. The induced pluripotent stem cell of claim 3 wherein the therapeutic entity is a microRNA Mir-302/367.

6. An anucleated erythrocyte comprising a therapeutic entity.

7. (canceled)

8. (canceled)

9. (canceled)

10. A pharmaceutical composition comprising an anucleated erythrocyte comprising a therapeutic entity, and a pharmaceutically acceptable carrier.

11. A method of treating or preventing cancer comprising the steps of:
administering to a subject thought to have cancer or prone of getting cancer an effective amount of, anucleated erythrocytes comprising a sc-mAb targeting human IGF1R (insulin-like growth factor 1 receptor); and
treating or preventing cancer in the subject.

12. A method of treating or preventing ischemic retinopathy comprising the steps of:
administering to the eye of a subject thought to have ischemic retinopathy or prone of getting ischemicretinopathy an effective amount of anucleated erythrocyte comprising a microRNA Mir-302/367, Myr-AKT (myristoylated, active form of AKT) or a combination thereof; and
treating or preventing ischemic retinopathy in the subject.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. A method of preparing anucleated erythrocytes comprising a therapeutic entity comprising the following steps:
providing induced pluripotent stem cells derived from a blood mononuclear cell that express a heterologous therapeutic entity protein or nucleic acid sequence under the control of an endogenous gene locus;
culturing the cells;
generating anucleated erythrocytes comprising the therapeutic entity; and
collecting the anucleated erythrocytes comprising the therapeutic entity.

25. A method of drug deliver comprising the following steps:
administering anucleated erythrocytes derived from an induced pluripotent stem cell derived from a blood mononuclear cell that expresses a heterologous therapeutic entity protein or nucleic acid sequence under the control of an endogenous gene locus to a subject.

26. A method of treating or preventing a cardiovascular disease comprising the steps of:
administering to a subject thought to have cardiovascular disease or prone of getting cardiovascular disease an effective amount of anucleated erythrocytes comprising a single-chain monoclonal antibody blocking proprotein convertase subtilisin/kexin type 9 (PCSK9); and
treating or preventing cardiovascular disease in the subject.

27. The induced pluripotent stem cell of claim 1 wherein the endogenous gene locus is the HBB gene.

28. The induced pluripotent stem cell of claim 1 wherein the induced pluripotent stem cell is human.

29. The method of claim 24 culturing the cells in E8 medium.

30. The method of claim 24 wherein the endogenous gene is the HBB gene.

34. The method of claim 25 wherein the endogenous gene locus is the HBB gene.

35. The anucleated erythrocyte of claim 6 is human and expresses hemoglobin.

36. An induced pluripotent stem cell derived from a blood mononuclear cell that expresses an exogenous therapeutic entity protein or nucleic acid sequence under the control of an endogenous gene locus.

37. The induced pluripotent stem cell of claim 36 wherein the endogenous gene locus is the HBB gene.
